(19) United States
(12) Patent Application Publication
(10) Pub. No.: US 2012/0296237 A1
(43) Pub. Date: Nov. 22, 2012

(54) MOTOR COORDINATION SKILLS MEASUREMENT AND ASSESSMENT

(76) Inventors: Gabriel Leonard, Montreal (CA); Brian Hynes, Anjou (CA)

(21) Appl. No.: 13/574,045

(22) PCT Filed: Jan. 20, 2011
(86) PCT No.: PCT/CA2011/000072

§ 371 Date: Jul. 19, 2012

Related U.S. Application Data
(60) Provisional application No. 61/296,745, filed on Jan. 20, 2010.

(51) Int. Cl. A61B 5/11

(52) U.S. Cl. 600/595

(57) ABSTRACT

There is described a computer-interfaced hand coordination measuring device that can track performance over time and report various types of errors, such as sequential, misses, perseverations, and balancing. The apparatus may be coupled to a computer and accompanying software is used to collect data from the administered tests and provide a report of the subject's scores for each hand on simple repetitive, sequential, and bimanual in-phase and out-of-phase tapping.

### CROSS-REFERENCE TO RELATED APPLICATIONS

This application claims priority under 35 U.S.C. §119(e) from U.S. Provisional Patent Application No. 61/296,745, filed on Jan. 20, 2010, the contents of which are hereby incorporated by reference.

### TECHNICAL FIELD

The present invention relates to the field of hand coordination measuring devices.

### BACKGROUND OF THE ART

The sophistication of the human motor system is best exemplified by how well we can coordinate our hand movements. For example, dexterous tool manipulation is needed in sports such as golf, in computer keyboard use, and in playing musical instruments. It has been shown that even small brain lesions can have devastating effects on motor coordination and movement sequencing, suggesting interruption of elegant neural connectivity.

Various tests may be administered to test the motor coordination of an individual. These tests are performed using a tapping apparatus, such as the one designed by Thurstone, which dates back to 1944, and is illustrated in FIG. 1. This apparatus allows the testing of unimanual and bimanual tasks in which subjects use a stylus to tap in a prescribed sequence on the board. On the bimanual tasks, the movements made by the two hands are out of phase. Tapping rates are determined by having a mechanical counter register a tap on the surface of the device and an operator visually confirm that the registered tap was received in the appropriate area.

Several drawbacks exist with the existing systems, such as the need to have an operator visually confirm that the tap is accurate, as well as the inability to test in-phase bimanual tasks. Therefore, there is a need for improvement to existing hand coordination measuring devices.

### SUMMARY

There is provided herein a computer-interfaced hand coordination measuring device that can track performance over time and report various types of errors, such as sequential, misses, perseverations, and balancing. The apparatus may be coupled to a computer and accompanying software is used to collect data from the administered tests and provide a report of the subject's scores for each hand on simple repetitive, sequential, and bimanual in-phase and out-of-phase tapping. The collected data may be compared with normative data which will act as the control data. Performances may be evaluated based on age, sex, weight, height, handedness, education, neurological condition, etc.

In accordance with a first broad aspect, there is provided a motor coordination measuring device comprising: a housing defining an enclosure and having a top surface composed of a first material; a pair of tapping targets on the top surface of the housing, each tapping target having at least two sub-targets, each of the at least two sub-targets identified by a symbol, the tapping targets composed of a second material different from the first material; and a tapping detection module inside the housing operatively connected to the tapping targets and adapted to record a tap when contact is made with the second material, the tapping detection module recording an instance of contact and differentiating between the sub-targets on each tapping target.

In accordance with a second broad aspect, there is provided a motor coordination testing system comprising: a motor coordination measuring device comprising: a housing defining an enclosure and having a top surface composed of a first material; a pair of tapping targets on the top surface of the housing, each tapping target having at least two sub-targets, each of the at least two sub-targets identified by a symbol, the tapping targets composed of a second material different from the first material; and a tapping detection module inside the housing operatively connected to the tapping targets and adapted to record a tap when contact is made with the second material, the tapping detection module recording an instance of contact and differentiating between the sub-targets on each tapping target; a computer operatively connected to the tapping detection module and comprising a software module adapted to receive tapping data, assess tapping performance, and output a tapping score.

In accordance with a third broad aspect, there is provided a computer system for assessing motor coordination skills using a device having a pair of tapping targets on the top surface of a housing, each tapping target having at least two sub-targets, each of the at least two sub-targets identified by a symbol, the system comprising: a processor; a memory accessible by the processor and comprising tapping data from the device; and a software module coupled to the processor, the software module configured for: receiving the tapping data; comparing the tapping data to a predetermined tapping sequence; generating a total test score; and outputting the total test score.

In accordance with a fourth broad aspect, there is provided a computer readable memory, for assessing motor coordination skills using a device having a pair of tapping targets on the top surface of a housing, each tapping target having at least two sub-targets, each of the at least two sub-targets identified by a symbol, the memory having recorded thereon statements and instructions which when executed by a computer will carry out the steps of: receiving tapping data from a motor coordination measuring device; detecting errors in the tapping data by comparison with a predetermined tapping sequence; generating a total test score in accordance with the detected errors; and outputting the total test score.

In this specification, the term “stylus” is intended to mean an elongated instrument used as an input device on a sensitive surface. A probe used to apply a voltage on a conductive surface is one type of stylus. Alternatively, the stylus may be a piece of molded plastic used to apply pressure to a pressure-sensitive surface, such as a touch screen or a touch plate. Various embodiments will be readily understood by those skilled in the art.

### BRIEF DESCRIPTION OF THE DRAWINGS

Further features and advantages of the present invention will become apparent from the following detailed description, taken in combination with the appended drawings, in which:

FIG. 1 is a schematic illustration of a motor coordination measuring device in accordance with the prior art;

FIG. 2A is a perspective view of a motor coordination measuring device in accordance with one embodiment;

FIGS. 2B to 2E are top schematic views of various exemplary housing shapes for the motor coordination measuring device;

FIGS. 3A to 3F are top schematic views of various exemplary configurations for tapping targets and sub-targets;

FIG. 4 illustrates the motor coordination measuring device of FIG. 1 without a top surface, in accordance with one embodiment;

FIG. 5 is a schematic diagram of a system to testing motor coordination skills, in accordance with one embodiment;

FIG. 6 is a block diagram of one embodiment of a system for assessing motor coordination skills;

FIG. 7 is a block diagram of one embodiment for the software module of FIG. 6; and

FIG. 8 is a flowchart illustrating one embodiment of a method for assessing motor coordination skills.

It will be noted that throughout the appended drawings, like features are identified by like reference numerals.

### DETAILED DESCRIPTION

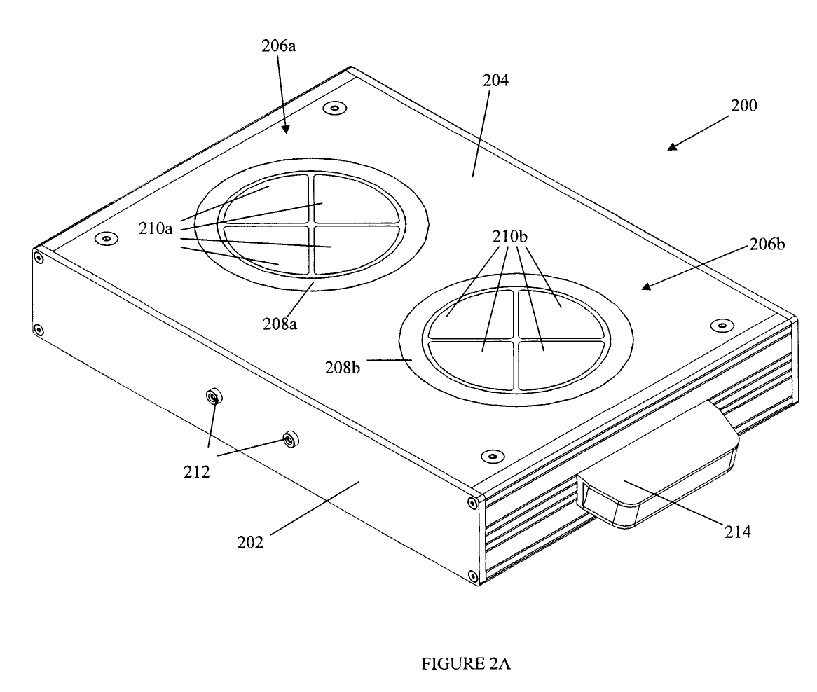

A perspective view of one embodiment of a motor coordination measuring device 200 is illustrated in FIG. 2A. A housing 202 defines an enclosure and is composed of four side walls, a bottom surface (not shown), and a top surface 204. In the embodiment illustrated in FIG. 2A, the housing 202 is rectangular-shaped and has a pair of connectors 212 provided along a sidewall of the housing 202 facing a pair of tapping targets 206a, 206b. When in operation, a set of styluses may be connected to the connectors 212, as will be described in more detail below. A handle 214 may be provided on the housing 202 to allow ease of transportation and manipulation of the motor coordination measuring device 200.

In other embodiments, the housing 202 may have an alternative shape, such as those illustrated in FIGS. 2B, 2C, 2D, and 2E. The connectors 212 may be provided along various positions of the housing, also illustrated in FIGS. 2B, 2C, 2D, and 2E. The connectors 212 may be on one or more of the sidewalls of the housing 202, or may be on the top or bottom surface thereof. In the case of a less practical positioning for the connectors 212, as seen in FIGS. 2D and 2E, a wire connecting each stylus to the device 200 should be long enough to allow manipulation of the stylus without disturbing the subject when the test is being administered. In another alternative embodiment, the stylus is wireless and no connectors are needed on the housing 202.

Referring back to the embodiment illustrated in FIG. 2A, the top surface 204 has a pair of tapping targets 206a, 206b, namely two circles, composed of sub-targets 210a, 210b in each tapping target 206a, 206b, i.e. four plates. In one embodiment, an annular plate 208a, 208b is also provided around each tapping target 206a, 206b and acts as an error ring. In one embodiment, the plates 210a, 210b, and the annular plates 208a, 208b, are made of a conductive material, such as steel, brass, copper, aluminum, or other types of metal that contain electric charges that will move when an electric potential difference is applied to the material. The top surface 204 is made of a non-conductive material, such as glass, wood, rubber, or plastic, such that no electricity passes through and a distinction is made between a conductive surface (on the plates) and a non-conductive surface (outside of the plates). In this embodiment, when the tip of a stylus, in the form of a powered probe, is applied to any one of the conductive surfaces, a circuit is closed and a voltage is applied to that surface.

In another embodiment, the tapping targets 206a, 206b may be touch screens, i.e. surfaces responsive to touch. A stylus may or may not be used to apply the touch to the surface. Various technologies may be used for the touch screens, such as resistive, surface acoustic wave (SAW), capacitive, projected capacitance, infrared, strain gauge, optical imaging, dispersive signal technology, acoustic pulse recognition, and coded LCD. In another alternative embodiment, touch plates are used. In this case, the surfaces of the tapping targets 206a, 206b are responsive to touch, either via a stylus or directly using a finger, and the detection circuitry is mechanical, such as resilient spring contact members rigidly mounted on a circuit board.

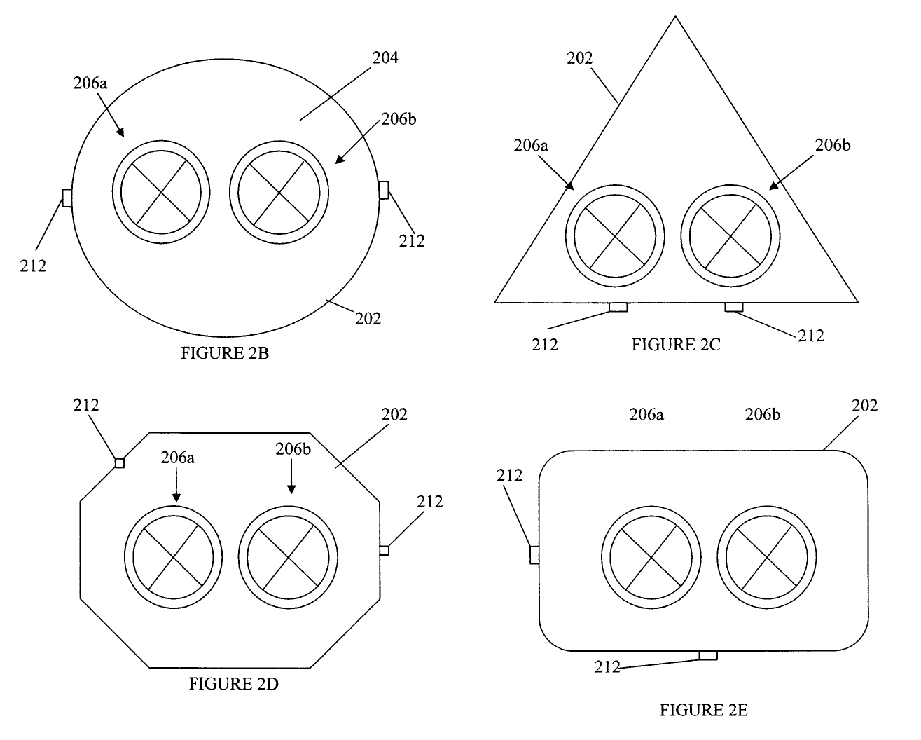

Alternative embodiments are also possible for the tapping targets 206a, 206b themselves. FIG. 3A illustrates a pair of circles as tapping targets 206a, 206b, with circle halves as each sub-target 208a, 208b. FIG. 3B illustrates a pair of circles as tapping targets 206a, 206b, with circle thirds as each sub-target 208a, 208b. FIG. 3C illustrates a series of concentric circles, each sub-target 208a, 208b being an area in between a pair of circles. In FIG. 3D, the tapping targets 206a, 206b, are square-shaped, and the sub-targets 208a, 208b are circles found inside each square. In FIG. 3E, the tapping targets 206a, 206b are square-shaped and the sub-targets 208a, 208b are quadrants of each square. In FIG. 3F, the square tapping targets 206a, 206b are sectioned into multiple strips, each strip representing a sub-target 208a, 208b. Further alternative embodiments will be readily understood by those skilled in the art.

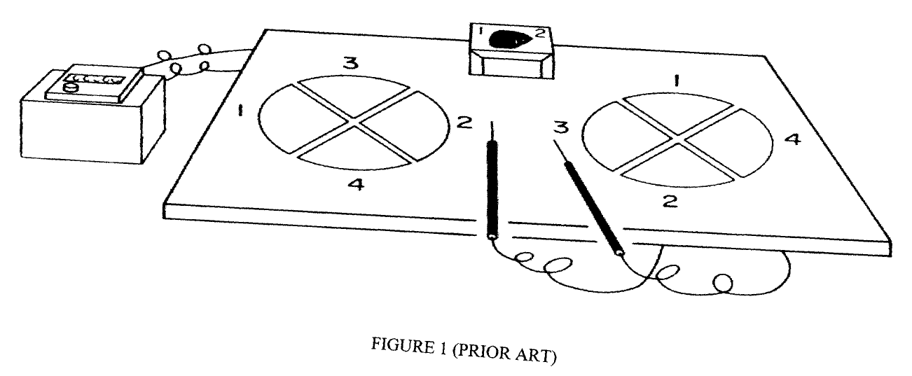

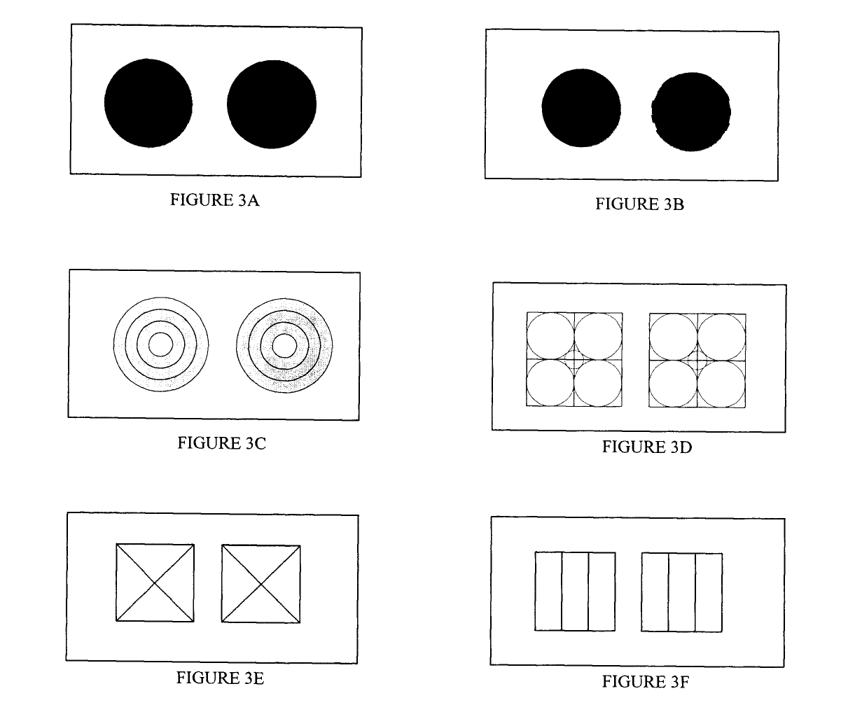

FIG. 4 illustrates the embodiment of FIG. 1 without the top surface 204. The housing 202 comprises a detection module 300, used to detect a tap on a sub-target 210a, 210b, of the tapping targets 206a, 206b. In the case of conductive plates, individual wires (not shown) may be provided from each plate 210a, 210b, 208a, 208b to the detection module 300. A debouncing circuit (not shown) may also be provided for each plate in order to remove rapid oscillations in the input signal that represent mechanical bouncing of a stylus on a plate rather than a valid tap. The module 300 reads the output of sensors which measure a voltage applied whenever at least one of the styluses comes into contact with one of the conductive plates. In a bimanual task, two simultaneous taps may be registered on different conductive plates. A counter may be used to increment an internal register each time a voltage pulse is detected at the counter input. This internal register is then read periodically to determine how many events have occurred.

In one embodiment, the detection module 300 is an off-the-shelf component known as Labjack™, which is a USB/Ethernet based measurement and automation device which provides analog inputs/outputs and digital inputs/outputs. An analog voltage signal may be read and registered, and then transformed into a digital signal. In an alternative embodiment, the detection module 300 is a detection circuit that corresponds to the technology used for the touch screen or the touch plate.

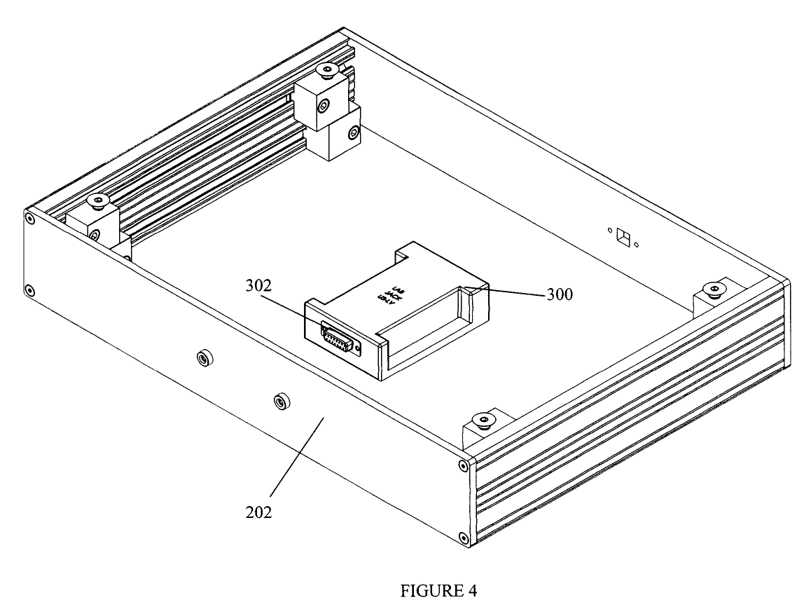

FIG. 5 is a schematic illustration of a system for testing motor coordination. As shown, each sub-target 210a, 210b of the tapping targets 206a, 206b are identified by a symbol. A sub-target 210a will have a matching symbol in a sub-target 210b, but not necessarily reflect a copy thereof. For example, the sub-targets 210a in FIG. 5 are sequentially identified as 1, 2, 4, 3 (clockwise) while the corresponding sub-targets 210b are sequentially identified as 4, 3, 2, 1 (clockwise). A variety of possible combinations for sub-target identification are possible. In addition, the symbols may be numbers, letters, shapes, images, etc. The symbols may also be of a same shape with differing colors.

In one embodiment, the sub-target identifications are provided on the annular plates 208a, 208b, and they may be changed. In one embodiment, a base annular plate 208a, 208b is permanently affixed to the top surface 204 of the motor coordination measuring device 200 and a new annular plate can be provided on top of the base annular plate using various attachment means such as screws, snaps, latches, etc. In another embodiment, the base annular plate is removable and may be replaced by a new annular plate using various attachment means such as screws, snaps, latches, etc. In an alternative embodiment (not shown), the identification symbols can be provided directly on the sub-targets 210a, 210b, which may themselves be removable or adapted to receive a new sub-target plate thereon. In another alternative embodiment (not shown) there is no annular plate and the identification symbols are provided directly on the top surface 204 of the housing 202. The identification symbols may be attached to the top surface 204 via releasable means such as Velcro™, tape, magnets, snaps, etc.

The motor coordination measuring device 200 is operatively connected to a computer 500. The computer 500 may be any programmable machine that can store, retrieve, and process data, such as a personal computer (PC), a laptop, a personal digital assistant (PDA), a server, etc. The motor coordination measuring device 200 collects the data and transmits it to the computer 500 for processing. A pair of styluses 502 are also illustrated. As indicated above, the styluses may require a physical connection to the motor coordination measuring device 200 or be wireless. They may be wireless due to a lack of any required power (i.e. completely made of a plastic material) or they may be battery-powered. In another alternative embodiment, the styluses harvest energy received via wireless means such as a Radio Frequency transmission and are remotely powered without the use of a battery or a physical connection.

The connection between the motor coordination measuring device 200 and the computer 500 may be wired or wireless. In one embodiment, a cable is connected from a USB port (302 in FIG. 4) on the detection device 300 to the computer 500. In other embodiments, other types of cable connections are possible. Wireless connections may also allow data collected by the detection module 300 to be transmitted to the computer 500 via a Local Area Network (LAN), a Metropolitan Area Network (MAN), a Wide Area Network (WAN), or any other type of network. The detection device 300 may transmit data to the computer 500 through radio-frequency, infrared, microwave, or other types of electromagnetic or acoustic waves. Any wireless technology used in cell phones, two-way radios, remote garage-door openers, television remote controls, and GPS receivers may be used. Various transmission protocols, such as WiFi, Bluetooth, and TCP/IP may be used. The data transmitted to the computer 500 may be in digital or analog form.

The motor coordination measuring device 200 may be powered by the computer 500, may be powered via a cable connection to a wall outlet, may be powered by an independent and external power supply, or may be battery-powered.

The computer 500 comprises a software module programmed to receive the data and process it accordingly. From the data, the location of the taps as well as the timing of each tap (or between taps) may be determined. The newly collected data is compared to normative data, which acts as control data, in order to assess performance.

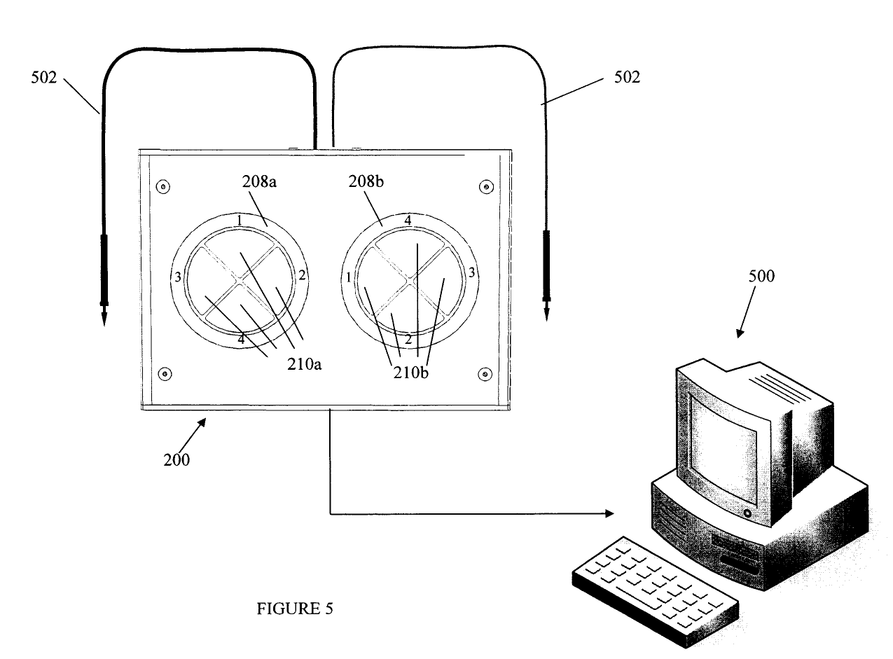

FIG. 6 illustrates an embodiment for computer 500. A memory 602 may store data such as received input data from the data detection module, predetermined sequences for various tests, normative data for comparison, and any other type of data or programs on a temporary or permanent basis. The memory 602 may be a main memory, such as a high speed Random Access Memory (RAM), or an auxiliary storage unit, such as a hard disk, a floppy disk, or a magnetic tape drive. The memory may be any other type of memory, such as a Read-Only Memory (ROM), or optical storage media such as a videodisc and a compact disc.

A processor 604 may access the memory 602 to retrieve data. The processor 604 may be any device that can perform operations on data. Examples are a central processing unit (CPU), a front-end processor, a microprocessor, a graphics processing unit (GPU/VPU), a physics processing unit (PPU), a digital signal processor, and a network processor. A software module 606, or an application, is coupled to the processor and is configured to perform various tasks.

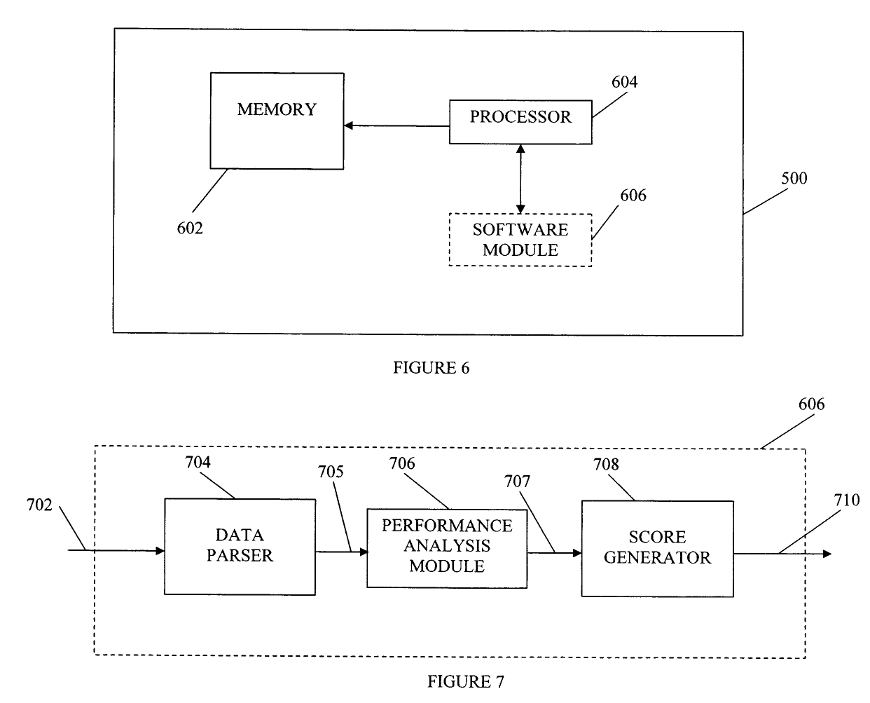

FIG. 7 is an exemplary embodiment of the software module 606. A data parser 704 receives the input data 702. When received, the input data may be in a digital format corresponding to a series of bits forming one or more words. In one embodiment, a “0” corresponds to a tap, or contact with one of the sub-targets/error rings, and a “1” corresponds to an absence of contact. The reverse is also possible. In one embodiment, the number of bits received corresponds to the number of sub-targets, and the position of each bit corresponds to one of the sub-targets. For example, using the motor coordination measuring device illustrated in FIG. 5, eight sub-targets and two error rings may be touched, which means 10 bits may be used, each bit representing either a sub-target or one of the error rings. If the error rings are wired together, a single bit may represent either error ring or two bits may be used but always set together at “1” or at “0”. Alternatively, every sub-target and both error rings are independent and have a corresponding bit.

Words may be scanned at an input port at any given rate, such as 1000 scans per second, 500 scans per second, or any other appropriate rate that corresponds to the type of testing taking place such that it will allow the collected data to be accurate. At each scan, a time stamp is assigned. In one embodiment, the data parser 704 will parse the data and eliminate repeat occurrences of words that do not indicate a change in status of the sub-targets/error rings, i.e. going from a contact to an absence of contact or vice versa. Once the parsing is completed, the parsed data 705 consists in one or more words representing a change in status and a corresponding time stamp for its time of occurrence. This data 705 is sent to a performance analysis module 706.

The performance analysis module 706 will receive the parsed data 705 and compare it with a predetermined sequence. The predetermined sequence corresponds to what the subject was asked to do as a testing task, such as 4 consecutive taps with only the left hand in a sequential manner (1-2-3-4) or a series of sequences with left and right hand in a predetermined order (2-4-1-3). Many different configurations are possible for the predetermined sequence.

Various tasks may be performed, and these include: unimanual sequential tapping trials (with each hand), out-of-phase and in-phase bimanual sequential trials, and simple repetitive unimanual tapping trials.

Unimanual sequential tapping involves the use of one hand to tap the numbered plates in numerical order (1-2-3-4) over and over as quickly as possible for fixed amount of time, such as 30 seconds. The unimanual task may be performed for each hand.

The in-phase bimanual sequential tapping task requires the subject to use both hands to tap the same sequence (1-2-3-4) on both of the numbered plates simultaneously, as quickly as possible for a fixed amount of time, such as 30 seconds. In order for this to be accomplished, the sub-targets of each tapping target are configured with similar configurations. The subject is asked to tap the 1-2-3-4 sequence with both hands, while making sure that the styluses hit the correct sub-targets at the same time (i.e. that the subject taps ‘1’ with the right hand and ‘1’ with the left hand, and there is a period of time during which both the left and the right styluses are in contact with the corresponding ‘1’ of each target, before moving on to ‘2’). If the subject taps the correct sequence on both targets but does not do so simultaneously, this is recorded as a ‘balancing’ error by the software module.

The out-of-phase bimanual sequential tapping task requires the subject to use both hands to tap sequences arranged differently on the sub-targets of each tapping target (again for a fixed amount of time, such as 30 seconds), while still attempting to tap simultaneously. The symbol identification configurations for the tapping targets used in this condition are different.

For simple repetitive unimanual tapping, the subject is asked to use one hand to tap on a single sub-target of a tapping target (plate ‘2’ on the right circle, or plate ‘4’ on the left) as quickly as possible for a fixed amount of time, such as 15 seconds. This task may be performed once with the right hand, and once with the left.

Performance may be evaluated based on how many errors are detected. In one embodiment, the tasks performed by the subject may result in five types of errors: 1) Sequential, the subject taps in an incorrect order (e.g. 1-2-4-3); 2) Perseverative, taps the same sub-target twice (in the sequential tasks); 3) Misses, recorded when the contact is made with the error ring surrounding the tapping targets; 4) Balancing, recorded when a left and a right tap occur simultaneously and each tap is on a plate that corresponds to the predetermined sequence, but the pair of taps together does not correspond to the predetermined sequence (i.e. 1 with left and 3 with right); and 5) Unimanual, recorded in a bimanual test when a tap is recorded with exactly one stylus and the tap is on a plate that corresponds to the predetermined sequence.

In one embodiment, the performance analysis module 706 will review the parsed data multiple times, and will look for a specific type of error during each pass. In another embodiment, the performance analysis module 706 will identify all errors using a single pass.

The performance analysis module 706 identifies errors 707 and sends them to a score generator 708. The data 707 sent to the score generator 708 may also include a total number of taps if this information is not already obtained by the score generator 708.

A tap may be defined as a press for a given hand followed by a release by that same hand. There are two possible taps for unimanual tasks: left hand press—left hand release; right hand press—right hand release. For a bimanual test, taps are identified for each hand separately. A unimanual tap is defined as a tap with exactly one hand, the other stylus released for the entire period of contact of the one stylus. A bimanual tap is defined as a pair of left- and right-hand taps that overlap temporally.

In one embodiment, the score generator 708 determines a score by subtracting a number of errors from a total number of taps. Other ways of generating a score will be readily understood, such as by considering a time factor for each task, or by cumulating the number of errors without considering a total number of taps.

Scores 710 may be compiled by the software module 606 and may be downloaded to a spreadsheet, stored in a database, or stored in any type of memory or recordable medium. Medical data and some questionnaire information may also be collected, either on-line or manually entered, and can be merged into the spread sheet and/or database with the tapping data/scores.

A tabulated summary of each participant's scores on each of the tasks may be presented. How many taps are made in time epochs may be tracked, for example the first 5 seconds versus the last 5 seconds. This latter capability allows fatigue effects in patient subjects and/or speed of learning in normal subjects to be examined. The timing of errors may also be tracked.

In one embodiment, the scoring data 710 is used to further assess the patient by comparing it with normative data. For example, if a random sample of subjects of a given age, a given gender, a given handedness, and a given height/weight are found to score X, a score above or below X may be indicative of various things, depending on the particular fact situation of the subject. For example, particular types of lesions on the brain may be typically present for a given range of scores. In another example, rehabilitation of a subject following an injury or an accident may be assessed in that a given range of scores may be indicative of a certain level of progress for the rehabilitation. Previous test results may be compared with new test results for a same patient to show progression or regression in motor coordination skills.

Subjects are asked to stand up and to position themselves in front of the motor coordination measuring device 200 (the height of the table is adjusted so that relative body position is matched for each subject's height). The operator may ask the subject to stand with their feet together, and to keep a non-tapping hand (in unimanual tasks) behind their back.

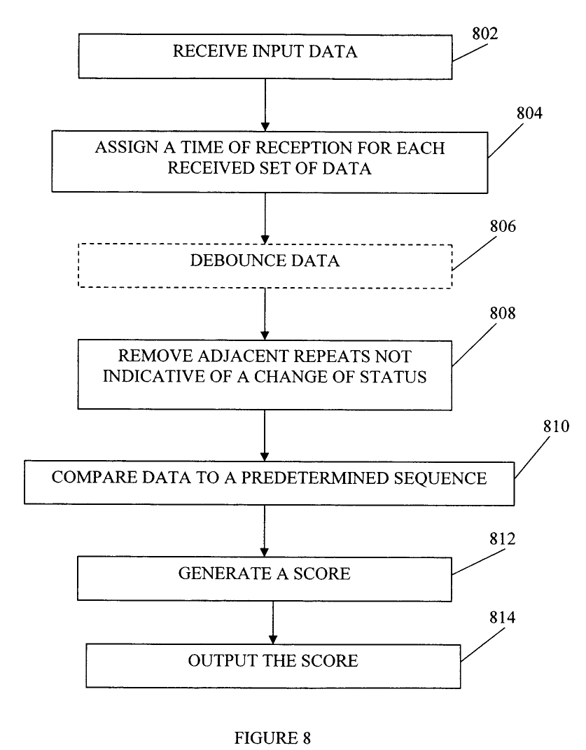

FIG. 8 is a flowchart illustrating a method for processing test data from a motor coordination test. The method may be performed by the system illustrated in FIG. 6. In a first step, the input data representing the test results from the motor coordination test is received 802. The time of reception for each set of input data received is assigned a time stamp 804. As indicated above, a set of input data may be a series of bits, such as 16 bits split into two words, or another number of bits.

As described above, a debouncing circuit may be present in the data detection module 300. Alternatively, the data may be debounced 806 in the software module 606 by applying the proper software debouncing routine. The data is also processed to remove adjacent repeats that are not indicative of a change of status 808 of the sub-targets/error rings. The analysis of the data then consists in comparing the data to a predetermined sequence 810, or to a desired or expected tapping rate. From this comparison, a score may be generated 812, and the score is then output 814 by the system.

It will be understood by those skilled in the art that the various embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment. It should be noted that the present invention can be carried out as a method, can be embodied in a system, a computer readable medium or an electrical or electro-magnetic signal. Additionally, while the present disclosure relates to code or functions that reside in a software module, this is not meant to limit the scope of possible applications of the described methods and module. Any system that utilizes static code on any type of computer readable medium, could be utilized without causing departure from the spirit and scope of the present disclosure.

The example embodiments of the present disclosure described above are intended to be examples only. Those of skill in the art may effect alterations, modifications and variations to the particular example embodiments without departing from the intended scope of the present disclosure. In particular, selected features from one or more of the above-described example embodiments may be combined to create alternative example embodiments not explicitly described, features suitable for such combinations being readily apparent to persons skilled in the art. The subject matter described herein in the recited claims intends to cover and embrace all suitable changes in technology.

1. A motor coordination measuring device comprising:
a housing defining an enclosure and having a top surface composed of a first material;
a pair of tapping targets on the top surface of the housing, each tapping target having at least two sub-targets, each of the at least two sub-targets identified by a symbol, the tapping targets composed of a second material different from the first material; and
a tapping detection module inside the housing operatively connected to the tapping targets and adapted to record a tap when contact is made with the second material, the tapping detection module recording an instance of contact and differentiating between the sub-targets on each tapping target.

2. The device of claim 1, wherein the first material is a non-conductive material and the second material is a conductive material.

3. The device of claim 1, wherein the tapping detection module is wired to each one of the at least two sub-targets and detects a tap when a voltage is applied to at least one of the at least two sub-targets.

4. The device of claim 1, further comprising an error zone composed of the second material and circumscribing each tapping target, wherein the tapping detection module is adapted to differentiate between the error zone and the sub-targets and record a tap in the error zone as a miss.

5. The device of claim 4, wherein the symbol identifying each sub-target is in the error zone.

6. The device of claim 5, wherein the error zone is detachable and may be replaced with another error zone having a different configuration for the symbols.

7. The device of claim 1, wherein each symbol identifying a sub-target is detachable and may be displaced to create a new configuration for the symbols.

8. (canceled)

9. (canceled)

10. The device of claim 1, wherein the tapping targets are circular and each contain four sub-targets representing a quarter of a circle, each quarter corresponding to an individual plate disposed on the top surface of the housing.

11. The device of claim 1, wherein the tapping detection module comprises a debouncing circuit to remove rapid oscillations from an input signal.

12. The device of claim 1, wherein the tapping detection module comprises a counter and an internal register, the counter incrementing the internal register each time a voltage pulse is detected, the internal register keeping count of how many events have occurred.

13. The device of claim 1, wherein the tapping detection module is adapted to detect simultaneous taps on the pair of targets and distinguish between the taps.

14. The device of claim 1, wherein the tapping detection module is adapted to detect a delay in taps of one of the tapping targets compared to the other one of the tapping targets.

15. A motor coordination testing system comprising:
a motor coordination measuring device comprising:
a housing defining an enclosure and having a top surface composed of a first material;
a pair of tapping targets on the top surface of the housing, each tapping target having at least two sub-targets, each of the at least two sub-targets identified by a symbol, the tapping targets composed of a second material different from the first material; and
a tapping detection module inside the housing operatively connected to the tapping targets and adapted to record a tap when contact is made with the second material, the tapping detection module recording an instance of contact and differentiating between the sub-targets on each tapping target; and
a computer operatively connected to the tapping detection module and comprising a software module adapted to receive tapping data, assess tapping performance, and output a tapping score.

16. The motor coordination testing system of claim 15, further comprising a pair of styluses for tapping the targets on the top surface of the motor coordination measuring device.

17. A computer system for assessing motor coordination skills using a device having a pair of tapping targets on the top surface of a housing, each tapping target having at least two sub-targets, each of the at least two sub-targets identified by a symbol, the system comprising:
a processor;
a memory accessible by the processor and comprising tapping data from the device; and
a software module coupled to the processor, the software module configured for:
receiving the tapping data;
comparing the tapping data to a predetermined tapping sequence;
generating a total test score; and
outputting the total test score.

18. The system of claim 17, wherein the software module receives the tapping data in digital format.

19. The system of claim 18, wherein “0” bits correspond to one of hits and misses and “1” bits correspond to the other of hits and misses.

20. The system of claim 19, wherein a number of bits received corresponds to a number of sub-targets, and a position of each bit in a word corresponds to a given sub-target.

21. The system of claim 20, wherein an additional bit is present for an error zone surrounding one of the tapping targets.

22. The system of claim 17, wherein the software module is further configured for parsing the tapping data and eliminating repeat occurrences of data that do not indicate a change in status of the sub-targets.

23. The system of claim 17, wherein the software module is configured to generate the total test score based on detected errors.

24. The system of claim 23, wherein detected errors are selected from a group consisting of sequential, perseverative, misses, balancing, and unimanual.

25. The system of claim 17, wherein the total test score includes a total number of taps.

26. The system of claim 17, wherein generating the total test score comprises taking into account time epochs to consider fatigue effects and/or speed of learning.

27. The system of claim 17, wherein generating the total test score comprises taking into account at least one of subject age, subject gender, subject handedness, and subject height/weight.

28. The system of claim 17, wherein generating the total test score comprises comparing a present score with a past score and assessing progress.

29. A computer readable memory, for assessing motor coordination skills using a device having a pair of tapping targets on the top surface of a housing, each tapping target having at least two sub-targets, each of the at least two sub-targets identified by a symbol, the memory having recorded thereon statements and instructions which when executed by a computer will carry out the steps of:
receiving tapping data from a motor coordination measuring device;
detecting errors in the tapping data by comparison with a predetermined tapping sequence;
generating a total test score in accordance with the detected errors; and
outputting the total test score.
